(19) United States
(12) Patent Application Publication
(10) Pub. No.: US 2008/0066205 A1
(43) Pub. Date: Mar. 13, 2008

(54) Method of Producing Young Moss Seedlings, Method of Producing Moss Mat and Young Moss Seedlings

(75) Inventors: Haruhiko Murase, Osaka-fu (JP); Kouichi Inoue, Osaka-fu (JP)

Correspondence Address: THE WEBB LAW FIRM, P.C., 700 KOPPERS BUILDING, 436 SEVENTH AVENUE, PITTSBURGH, PA, 15219, US

(73) Assignee: Hero Corporation, Osaka-fu (JP)

(21) Appl. No.: 10/566,326

(22) PCT Filed: Feb. 9, 2005
(86) PCT No.: PCT/JP05/01957

§ 371 Date: Jan. 27, 2006

(30) Foreign Application Priority Data
Feb. 10, 2004 (JP) 2004-033525
Jul. 26, 2004 (JP) 2004-217231

(51) Int. Cl. A01H 4/00; A01G 31/00; A01H 11/00

(52) U.S. Cl. 800/295; 47/58.1 SE; 47/59 R; 47/59 S

(57) ABSTRACT

The present invention relates to a method of producing young moss seedlings preferable as greenery plants, by which a plenty of young moss seedlings can be produced with high speed under artificially controlled atmosphere. In nutrient solution, young moss seedlings are grown with regeneration buds 2 bred around gametophytes 1, at a temperature of 0 to 60° C., with photosynthetic active photon flux density (PPFD) of not greater than 200 (μmolm⁻²s⁻¹), fertilizer concentration (mS/cm) of 0 to 1.0, and by repeating light periods and dark periods in cycles of 24 hours or less duration, aerating and stirring, and controlling growth of young moss seedlings.

### FIELD OF THE INVENTION

The present invention relates to a method of producing young moss seedlings, in particular, for young moss seedlings preferable as greenery plants, by which a plenty of young moss seedlings can be produced with high speed, to a method of producing a moss mat in which moss is prepared in a mat-like state for convenient construction, and to young moss seedlings.

### BACKGROUND OF THE INVENTION

Usually, moss is cultivated under artificially natural atmosphere as a main material for gardening. This can be done because demand for moss is relatively little and therefore, cultivation under natural atmosphere can fully meet the demand. Moss is grown taking a long time of 2 to 3 years since the growth thereof is slow under the present technology.

Since moss prefers particular growth atmosphere, it is grown in few utilizable places as cultivation lands. Therefore, conventionally, some part of moss is shipped as landscape live moss and other part thereof is shipped as cut and dried moss species during production processes.

On the other hand, as a greenery plant, construction is being carried out with moss mats in which moss is prepared in mat-like covering and fixing on roofs of buildings, wall surfaces, road side walls, shore protection surfaces, and the like. Moss is resistant to climate changes as a greenery plant and grows without soil, and therefore, said condition is preferable since moss is light weighted and requires substantially no maintenance. A moss mat is a mat-like material, where moss grows in a cluster like a so-called grassy mat, and usually from the view point of handling moss for construction purposes and the like, a moss mat is defined as a moss got tangled in a substantially 40 to 60 cm-square on which buds of moss (ear-like ones) grow out on the upper surface of the mat. In general, a moss mat is produced by putting young moss seedlings in a base plate composed of a net with resin fibers knitted, one whose enveloping surface is plate-like with filament tangled, a resin-made frame by injection molding and the like, or combinations thereof, followed by a process called a rearing process in which moss is proliferated and length of moss is made to grow.

In order to produce moss mats, a large amount of moss is required, however, the actual condition is that production technology of moss has not yet been established. In particular, Racomitrium canescens which has strong resistance against dryness and which grows even on inorganic plates such as wall surfaces of buildings is preferable for a greenery plant in this regard, however, under natural environment, it grows very slow, therefore, it has been difficult to utilize as a greenery plant which requires mass cultivation.

Conventionally, technology has been proposed which uses moss as a greenery plant (Patent document 1). In said patent document 1, a method for repeatedly subculturing a gametophyte of moss indigene native to fields in a multidimensional manner by obtaining the gametophyte or a method for cultivating cells of moss plants by using culture medium has been proposed. (Patent document: page 4, column 7, line 29 to page 4, column 8, line 20).

Patent document 1: U.S. Pat. No. 2,863,987

However, as these technologies take time and trouble, such technologies that produce a large amount of moss with high speed under artificially controlled environment have not always been provided. Further, even when young moss seedlings come out, in order to prepare moss mats convenient for construction, rearing of the young moss seedlings is necessary, that is, to let young moss seedlings proliferate in a moss mat support medium, and allow rooting to grow in the direction of the half side of a mat. A mat body is reared under natural environment or under controlled environment with roofs and the like with water sprinkled to expedite moss growth. Since it takes several years or months for this rearing, it has also been a bottleneck on utilizing moss for greening.

The object of the present invention is to provide a method for producing a large amount of young moss seedlings under artificially controlled environment with high speed, and in particular, a method for producing young moss seedlings of Racomitrium canescens the like suitable for greenery plants, and a method for producing moss mats in a short time.

### SUMMARY OF THE INVENTION

As a result of intensive studies for solving the above problems, the inventors have found the unexpected phenomena that a large amount of regeneration buds (lateral buds) breed with high speed around gametophytes of moss which have initially been considered to wither away and die down when young moss seedlings are grown in nutrient solution without finding for growing environment of moss in natural environment, thereby completing the present invention based on this finding.

The present invention relates to a method of producing young moss seedlings characterized in that young moss seedlings are grown in nutrient solution and a method of producing moss mats in which moss mats are produced in nutrient solution. In other words, the present invention relates to a method of producing moss controlling the growth of moss in nutrient solution.

In particular, young moss seedlings are preferably grown in nutrient solution within the range of a temperature of 0 to 60° C. and of a photosynthetic active photon flux density of not greater than 200 (μmolm⁻²s⁻¹).

Further, it is preferable that gametophytes of moss are grown letting gaseous body including oxygen (e.g. air and the like) intermittently contact with said gametophytes in nutrient solution by, for example, aerating and stirring. In particular, when young moss seedlings are grown in nutrient solution with aeration and stirring, since young moss seedlings are grown stirred in nutrient solution, impact of light and gravity can be freely imparted from the 360-degree direction without limited to a certain direction, and regeneration buds can be bred around gametophytes, thereby capable of obtaining young moss seedlings preferable for greening. By this, for example, young moss seedlings in which enveloping surfaces at the tip parts of said bred regeneration buds are spindle-shaped preferable for greening can be produced.

In addition, young moss seedlings can also be grown by fixing said young moss seedlings in a certain direction in nutrient solution and also, young moss seedlings having regeneration buds with breeding directionality around gametophytes can be cultivated. Thus, since young moss seedlings are grown in nutrient solution, the positions of young moss seedlings in nutrient solution can be controlled and therefore, breeding forms suitable for various uses such as greenery uses can be imparted to young moss seedlings.

In addition, it is desirable that young moss seedlings are grown by repeating light periods and dark periods in cycles of 24 hours or less duration and that they are grown under low concentration of fertilizers of, for example, 0 to 1.0 in electric conductivity (mS/cm) in nutrient solution.

Further, like the above, young moss seedlings can be grown in nutrient solution in a state where the above mentioned young moss seedlings or gametophytes of moss are retained on a mat support medium having an aperture portion at least on one surface, and further, by growing moss buds which outgrow from the aperture portion of a mat support medium in nutrient solution in the normal line direction, rearing of a moss mat can be conducted in a short time.

### BRIEF EXPLANATION OF THE DRAWINGS

FIG. 1 is a view showing an image with 0 day passing from the onset of moss piece cultivation.

FIG. 2 is a view showing FIG. 1 in the form of a diagram.

FIG. 3 is a view showing an image with 7 days passing from the same cultivation.

FIG. 4 is a view showing FIG. 3 in the form of a diagram.

FIG. 5 is a view showing an image with 21 days passing from the same cultivation.

FIG. 6 is a view showing FIG. 5 in the form of a diagram.

FIG. 7 is a view showing an image with 50 days passing from the same cultivation.

FIG. 8 is a view showing FIG. 7 in the form of a diagram.

FIG. 9 is a view showing a frame format of a cultivation state of Example 2.

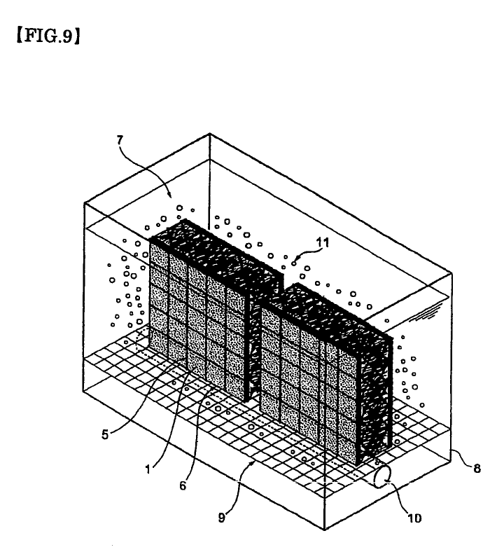

### DETAILED DESCRIPTION OF THE EMBODIMENTS

Although moss used in the present invention is not specifically limited and regardless of mycorrhiza or sterile, any moss may be used so long as its regeneration bud breeds around a gametophyte grown in nutrient solution. For example, Bryopsida and Hepaticopsida may be used and in particular, a moss plant of Bryopsida is preferable. For example, in Bryopsida, Rhacomitrium Bird. including Racomitrium canescens, Racomitrium ericoides, Racomitrium japonicum, Racomitrium lanuginosum, Racomitrium heterostichum, Racomitrium laetum, Rhacomitrium sudeticum, Racomistrium fasciculare, Rhacomitrium anomodontoides, Racomistrium fasciculare, Rhacomitrium carinatum, Rhacomitrium aciculare, and the like, Thuidium B.S.G including Thuidium kanedae, Thuidium cymbifolium, Thuidium tamariscinum, Thuidium delicatulum, Thuidium recognitum, Thuidium pristocalix, Thuidium sparsifolium, and the like, Climacium Web et Mohr including Climacium japonicum, Climacium dendroides, and the like, Dicranum Hedw. including Dicranum scoparium, Dicranum japonicum, Dicranum nipponense, Dicranum fuscescens, Dicranum majus, Dicranum caesium, Dicranum polysetum, Dicranum drummondii, Dicranum flagellare, Dicranum mayrii, Dicranum viride, Dicranum fulvum, Dicranum hamulosum, Dicranum leiodontum, and the like, Hypnum Hedw. including Hypnum plumaeforme, Hypnum sakuraii, Hypnum oldhamii, Hypnum calcicolum, Hypnum fujiyamae, Hypnum cupressiforme, Hypnum tristo-viride, Hypnum pallescens, Hypnum reptile, Hypnum plicatulum, Hypnum subimponens, Hypnum erectiusculum, Hypnum lindbergii, and the like, Rhizogonium Brid. including Rhizogonium dozyanum, Rhizogonium spiniforme, Rhizogonium spiniforme, and the like can be used.

In Hepaticopsida, moss which belongs to Heteroscyphus Schiffn. including Heteroschyphus planus, Heteroscyphus argutus, Heteroschyphus bescherellei, Heteroscyphus lophocoleoides, Heteroscyphus tener, Chiloscyphus aselliformis, and the like, Porella. L including Porella perrottetiana, Porella stephaniana, Porella campylophylla, Porella caespitans, Porella japonica, Porella oblongifolia, Porella grandiloba, Porella vernicosa, Porella fauriei, Porella gracillima, and the like, Bazzania S. Gray including Bazzania japonica, Bazzania yoshinagana, Bazzania fauriana, Bazzania trilobata, Bazzania denuadata, Bazzania bidentula, Bazzania tricrenata, Bazzania pearsonii, Lepidozia pompeana, Bazzania tridens, Bazzania mayebarae, and the like can be used.

In particular, Rhacomitrium Canescens including Racomitrium canescens, Racomitrium ericoides, Racomitrium japonicum, and the like are preferable for greening of buildings. This moss requires no soil or fertilizer for growth and can survive in very dried condition without putting excess burden on wall surfaces of buildings, for example. Although growth speed of this kind of a young seedling is very slow under natural environment, in the present invention, it can be cultivated in mass with high speed.

Material moss (material seedling) grown in nutrient solution can be obtained by collecting gametophytes, in particular, gametophytes having forms of leafy gametophytes from moss indigene native to fields or commercially available one.

Temperature of nutrient solution is 0 to 60° C., preferably 5 to 50° C., and further preferably 15 to 25° C. When temperature of nutrient solution is less than 0° C., it is not preferable since the nutrient solution freezes. When temperature of nutrient solution exceeds 60° C., microbes and the like is likely to breed, which slows moss growth, and therefore, it is not preferable, either.

Concentration of a fertilizer of nutrient solution is 0 to 1.0 in electric conductivity (mS/cm) and preferably 0 to 0.2, and nutrient solution preferably includes a nutrient solution with low concentration or no fertilizer included. As a fertilizer, a general fertilizer (for example, HYPONeX®) can be used. When concentration of a fertilizer exceeds 1.0 (mS/cm), since it is too thick as one, growth of a young moss seedling is likely to slow and therefore, it is not preferable.

Nutrient solution preferably includes phytohormone. As phytohormone, ethylene, abscisic acid, auxin (indoleacetic acid), cytokinin (zeatin), gibberellin (gibberellic acid), and the like can be exemplified, however, plant growth hormone is preferable and among them, auxin, cytokinin, and gibberellin are preferable. Gibberellin is the most preferable for large growth promoting effect. They may be used alone, may be used in mixtures and/or may be used together. When concentration of plant growth hormone is high, it causes physiological disorder of a plant, therefore, concentration of 0.01 to 0.2 ppm is preferable, diluted with about 1000 times compared with concentration used for general gardening, and concentration of 0.1 ppm is further preferable.

Light is required for growth of moss. Light may be natural or artificial and light source is not specially limited, however, it is necessary that the light has ingredients of wavelength regions which enables photosynthesis and which do not inhibit growth. When growing moss under artificial environment, a general incandescent lamp, fluorescent lamp, mercury lamp, LED, and the like can be used, however, LED is preferable from the view point of low energy cost, easy heat control, and easy maintenance, and among them, LED which emits red light, or orange light is preferable. When LED is incorporated in a moss cultivating device, use of LED arranged in the form of a two-dimensional array will make it compact. Therefore, it becomes easy to prepare a cultivation container which has a flat bubbler connected to an air pipe in which nutrient solution and gametophytes of moss are put and an LED array as one unit, and said unit is piled up three-dimensionally in multiples. An LED array is preferable for a long life due to low electric energy and little waste heat.

Photosynthetic active photon flux density(PPFD)is preferably not greater than 200 (μmolm⁻²s⁻¹), particularly preferably not greater than 50 (μmolm⁻²s⁻¹), and further preferably 20 to 30 (μmolm⁻²s ¹) which is not less than light compensation point. When photosynthetic active photon flux density is high, it is not preferable since growth of young moss seedlings is likely to slow. For information, when young moss seedlings are grown in nutrient solution, it is preferable to dim a light by repeating light and dark periods. In particular, it is preferable that young moss seedlings are grown by repeating light periods and dark periods in cycles of 24 hours or less duration. By repeating light periods and dark periods at specified cycles, the balance between photosynthesis and metabolism are secured and therefore, it is preferable. Therefore, for example, by setting the cycle time every 12 hour initially and then every 6 hour after a passage of a specified time, for example, the cycle of light periods and dark periods depending on the growth stages of young seedlings is controlled, thereby capable of controlling breeding amount and speed of regeneration buds.

The direction of regeneration buds generated by light stimulation is affected by gravitation, light direction, oxygen concentration, and the like. The growing direction of lateral buds has positive photoaxis. When light is emitted to moss gametophytes suspended in nutrient solution from 360° direction, regeneration buds are generated in every direction. For this reason, young moss seedlings in which an enveloping surface at the tip of said bred regeneration buds are spindle-shaped are generated. On the other hand, since growing direction of regeneration buds can be controlled by the light direction, when the light is emitted from the direction where lateral buds are desirably grown in designing moss mats, regeneration buds grow in the incidence direction of the light.

When gametophytes of moss are filled in a moss mat support medium and are grown in nutrient solution in a state where free motion of gametophytes is prohibited, light is emitted from the vertical direction to an aperture portion of a moss mat support medium and regeneration buds are generated toward the aperture portion. And by taking said regeneration buds as they are out of nutrient solution, further followed by rearing them with light emitted from the vertical direction to an aperture portion of a moss mat support medium, a moss mat can be produced in which so-called moss ears are grown vertically to its surface from a mat aperture portion.

On the other hand, when gametophytes of moss are filled in a moss mat support medium and are grown in nutrient solution in a state where free motion of gametophytes is prohibited, light is emitted from the vertical direction to an aperture portion of a moss mat medium and regeneration buds are generated toward the aperture portion. And by keeping on cultivating in nutrient solution, a moss mat can be produced at once in which tips of regeneration buds are grown vertically to its surface from a mat aperture portion like a so-called moss ears.

In addition, in the method of the present invention, it is important to let a gaseous body which includes oxygen contact with gametophytes of moss in nutrient solution intermittently by for example aerating and stirring, thereby making them grow, while stirring.

By this, regeneration buds which used to wither away or die down in liquid breed and proliferate. Although methods of stirring by bubbling the gaseous body such as air and the like are not limited to aerating and stirring, aerating and stirring are preferable as a convenient method.

For information, devices for carrying out the method of the present invention, for adjusting environment at a constant temperature and constant light, a complete control type is preferable and by the complete control type, previous step of plant factories preferable for mass production can be made up. Therefore, when young seedlings of moss obtained by the above method are transferred to a medium for a moss mat under a plant factory environment and reared, a large amount of regeneration buds bred and proliferated from a large amount of gametophytes are further grown under controlled environment of a plant factory, in particular, under computer control, and therefore, a large amount of moss community can be produced, which can formulate a system as a production factory of a series of greenery moss plants.

### EXAMPLES

### Example 1

A sample was prepared using a moss piece (15 mm) obtained by cutting a part of wild Racomitrium canescens (gametophytes with leafy gametophytes) obtained from mountain fields in Izumi-shi, Osaka-fu, and 25 g of the sample was put in a 500 ml culture tank, followed by stirring by bubbling air under completely controlled environment with 50 (μmolm⁻²s⁻¹)of photosynthetic active photon flux density (PPFD) by a fluorescent light, 12 hours of light and dark periods, 15° C. of nutrient solution temperature, and HYPONeX® with fertilization concentration of 0.2 (mS/cm), thereby cultivating young seedlings.

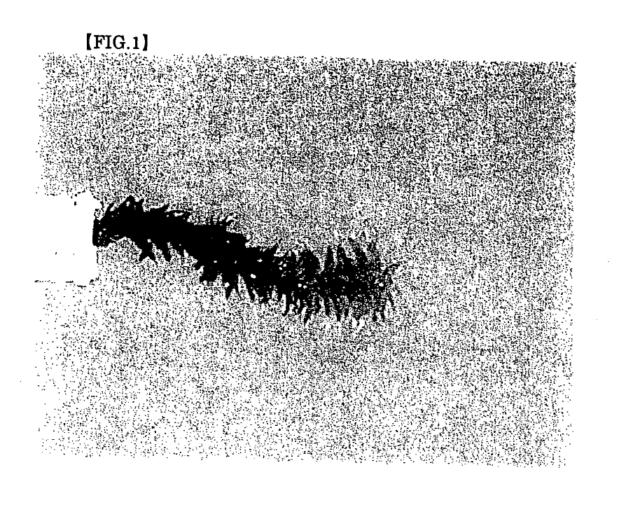

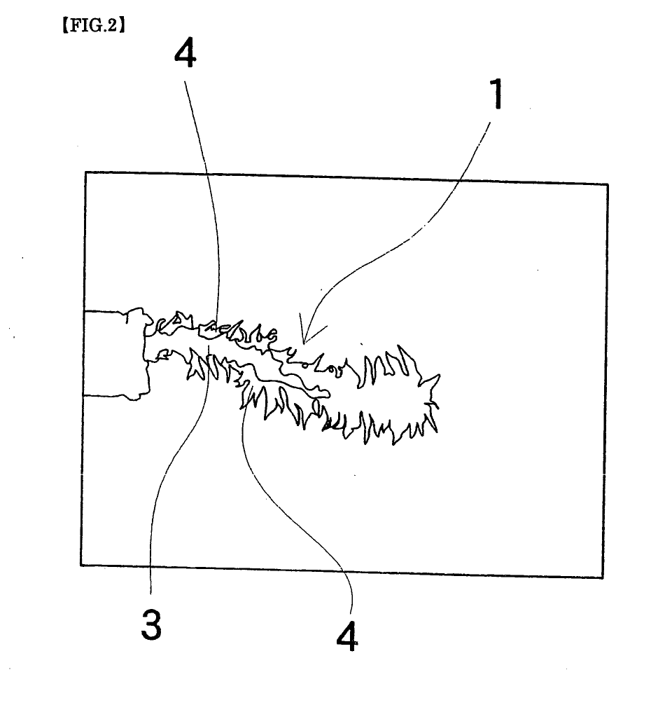

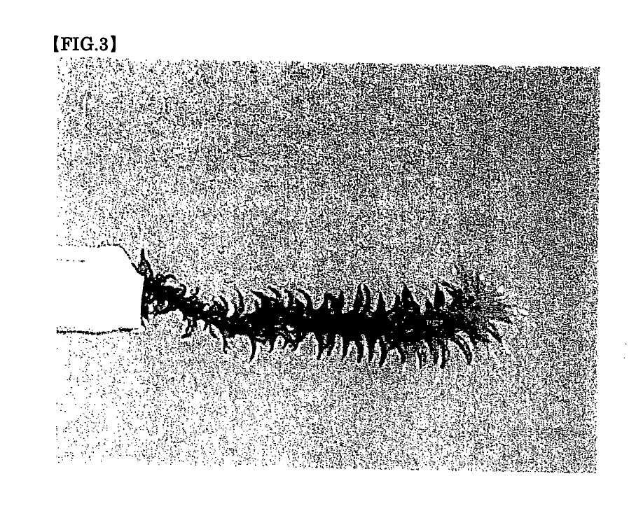

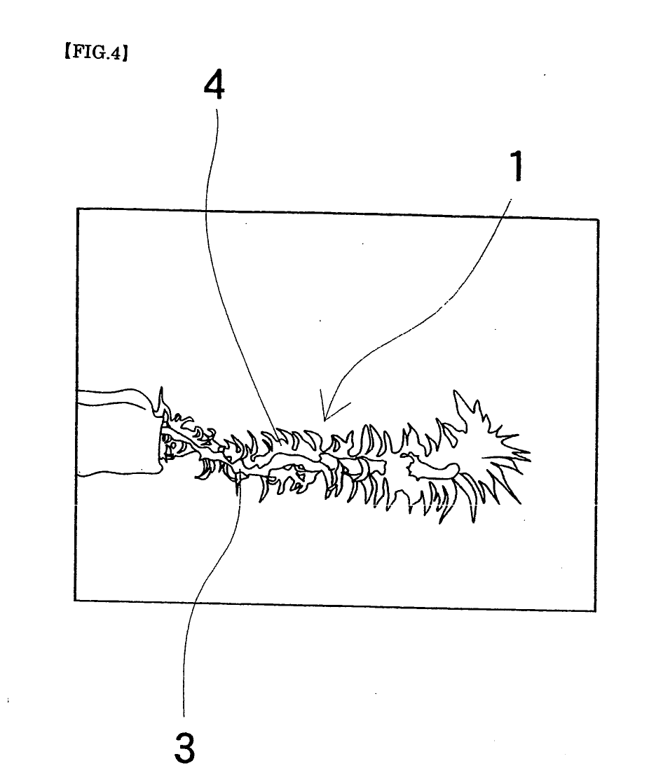

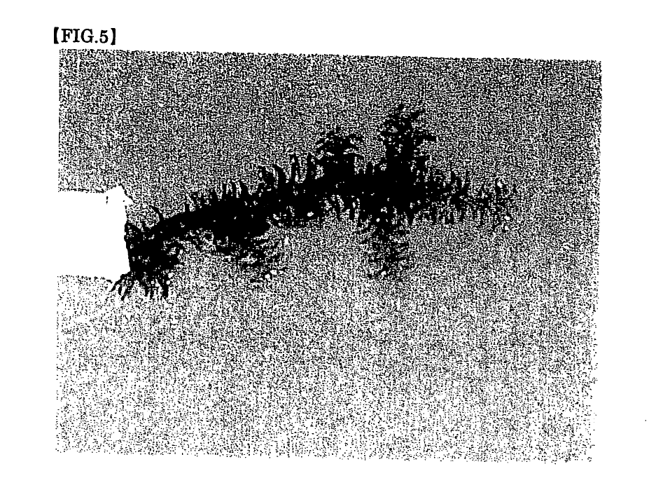

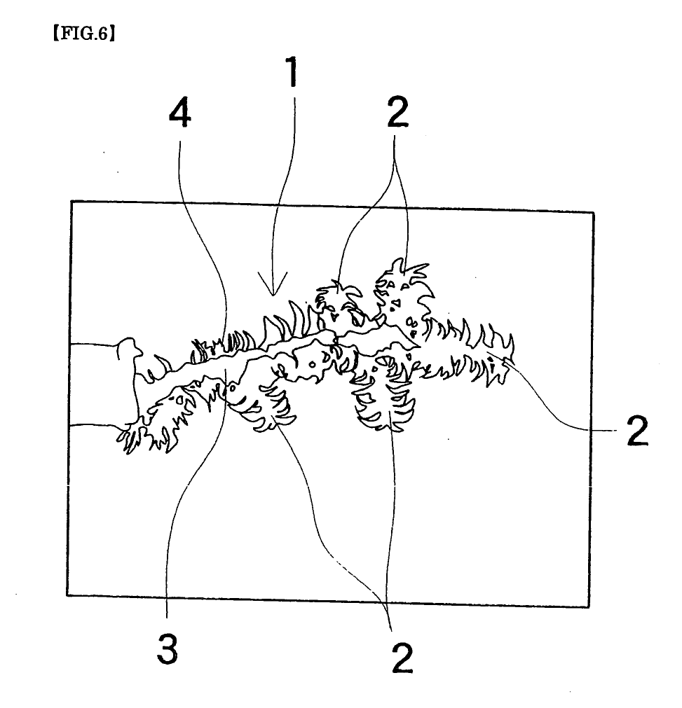

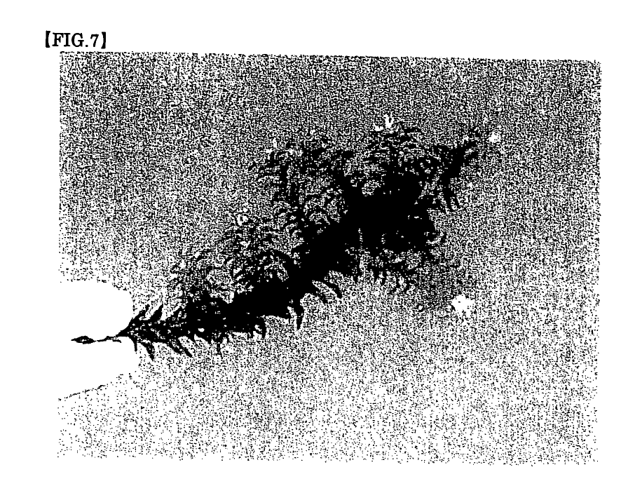

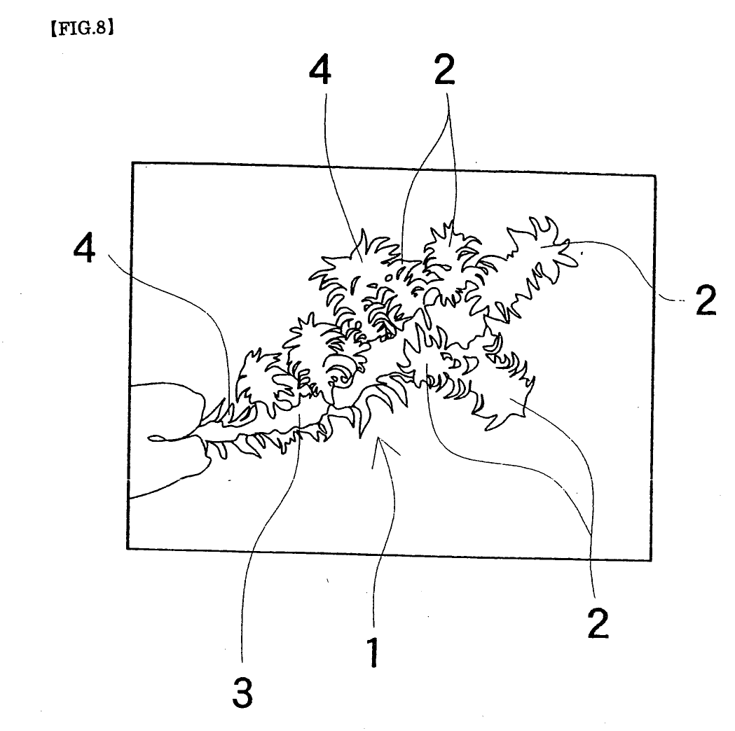

FIG. 1 is a view showing an image with 0 day passing from the onset of moss piece cultivation, FIG. 2 is a view showing FIG. 1 in the form of a diagram, FIG. 3 is a view showing an image with 7 days passing from the same cultivation, FIG. 4 is a view showing FIG. 3 in the form of a diagram, FIG. 5 is a view showing an image with 21 days passing from the same cultivation, FIG. 6 is a view showing FIG. 5 in the form of a diagram, FIG. 7 is a view showing an image with 50 days passing from the same cultivation, and FIG. 8 is a view showing FIG. 7 in the form of a diagram. Every image diagram shows moss pieces enlarged by about three times image-processed on a computer after digitally photographing the moss pieces.

As shown in FIG. l (FIG. 2), in gametophyte 1 with 0 day passing from the onset of cultivation, only leafy gametophyte 4 is around a leaf stalk 3, while on the other hand, as shown in FIG. 5 (FIG. 6) and FIG. 7 (FIG. 8), from 21 days to 50^{h} days passing from the onset of cultivation, plural of or multiple of regeneration buds 2 clearly breed and proliferate around gametophyte 1. It is considered that regeneration buds 2 breed from a withered or dormant leaf stalk 3. By this, according to such a method, young moss seedlings suitable for greenery plants in which regeneration buds 2 breed all around the moss gametophyte 1.

Under the above mentioned environmental condition, the growth speed of each young moss seedling was about 5 mm in 3 weeks. This is about 8 times as fast as the growth speed under natural environment.

In addition, in the case young moss seedlings are produced as a front-end of the plant factory environment, and when a production method as mentioned above is employed, for example, since the design can be employed in proportion as the capacity of a culture tank with regard to culture amount, young seedlings equivalent to 0.25 m² can be grown in a 5 litter tank.

### Example 2

A casing 6 having a parallelepiped stereoscopic structure was formed by filling moss pieces 1 (average length of 15 mm) as used in Example 1 densely in a polyolefin stereoscopic net 5 for a moss mat of 5 cm in length, 5 cm in width, and 1.5 cm in thickness which is a moss mat support medium stereoscopically woven with polyethylene fibers, thereby restricting the mutual motion of moss pieces. Two casings in total together with other casing 6 manufactured by the same method were fixed on an air-permeable partition wall 9 in a culture container 8 by dipping the lateral surface of the above mentioned casings in a vertical direction in a transparent culture container 8 filled with nutrition solution 7 of HYPONeX® diluted by 1000 times.

Cultivation was continued, pumping air from an air supply tube 10, letting air bubble 11 generate so that enough amount of air can be supplied from a flat-type bubbler (trade name “AIRSTONE” not illustrated) attached to the air supply tube 10 to the casing 6, stirring nutrient solution by bubbling, irradiating light so that an irradiated surface on which moss pieces are filled in polyolefin stereoscopic net 5 intermittently every 12 hour from the front face of a cultivation container has PPFD of 50 μmolm⁻²s⁻¹ and maintaining liquid temperature at 15° C. Moss growth was substantially the same as in Example 1 in which moss alone was subject to hydroponic cultivation.

### Example 3

One of the casings of Example 2 was taken out from the cultivation container 3 weeks after the onset of cultivation and was put on a concrete sprinkling water from time to time as in ordinary rearing, rearing to be exposed to sunlight above thereby obtaining a moss mat.

### Example 4

Further cultivation was continued for 5 weeks regarding the other remaining casing. A moss mat was produced in which the tip of moss regeneration buds began to grow by 5 mm from an aperture portion on a light irradiated net surface.

### Example 5

Young moss seedlings were cultivated as in Example 1 except that gibberellin was added with the concentration of 0.1 ppm in nutrient solution. The growth speed of young moss seedlings was about 5 mm in 1 week, respectively.

### EFFECT OF THE INVENTION

By this, when gametophytes are obtained from moss indigene native to fields and the gametophytes are put in a tank in which nutrient solution is stored, for example, regeneration buds are bred and proliferate around the gametophytes of moss with high speed, and therefore, a large amount of moss can be produced with high speed. And by growing young moss seedlings under controlled environment, moss community suitable for greening can be provided.

Further, by rearing moss mat bodies in nutrient solution, moss mats convenient for construction can easily be produced in a short time. Young moss seedlings with peculiar forms in which regeneration buds proliferate around the gametophytes of moss are obtained.

### INDUSTRIAL APPLICABILITY

According to the method of the present invention, since a large amount of young moss seedlings can be produced with high speed under artificially controlled environment, plant factory system for growing moss including young seedling cultivation facilities can be built up. Further, since a large amount of moss mats can be produced with high speed, moss plants can be put to practical uses for the first time as greenery plants in construction fields or environment-related fields including greening of roofs, wall surfaces, and the like. In addition, young moss seedlings obtained by the present production method can be applied to various purposes such as gardening, landscaping, medical purposes and the like not only to the purpose of greenery plants.

What is claimed:

1. A method for producing young moss seedlings wherein moss is grown in nutrient solution.

2. The method of production as set forth in claim 1, wherein regeneration buds are bred around gametophytes of moss in nutrient solution.

3. The method of producing young moss seedlings by growing the young moss seedlings letting a gaseous body which includes oxygen contact with the gametophytes of moss intermittently within the range of a temperature of 0 to 60° C., and photosynthetic active photon flux density (PPFD) of not greater than 200 (μmolm⁻²s⁻¹), respectively.

4. The method of producing young moss seedlings by growing the young moss seedlings, aerating and stirring within the range of a temperature of 0 to 60° C., and photosynthetic active photon flux density (PPFD) of not greater than 200 (μmolm⁻²s⁻¹), respectively.

5. The method of producing young moss seedlings as set forth in claim 3, wherein moss is grown by repeating light periods and dark periods in cycles of 24 hours or less duration.

6. The method of producing young moss seedlings as set forth in claim 4, wherein moss is grown by repeating light periods and dark periods in cycles of 24 hours or less duration.

7. The method of producing young moss seedlings as set forth in claim 3, wherein fertilizer concentration of said nutrient solution is 0 to 1.0 (mS/cm).

8. The method of producing young moss seedlings as set forth in claim 4, wherein fertilizer concentration of said nutrient solution is 0 to 1.0 (mS/cm).

9. The method of producing young moss seedlings as set forth in claim 1, wherein said nutrient solution includes phytohormone.

10. The method of producing young moss seedlings as set forth in claim 9, wherein said phytohormone includes at least one from gibberellin, cytokinin, and auxin.

11. The method of producing young moss seedlings as set forth in claim 1, wherein said young seedlings are moss plants of Bryopsida.

12. The method of producing young moss seedlings as set forth in claim 11, wherein said moss plants are Racomitrium canescens.

13. The method of producing young moss seedlings as set forth in claim 1, wherein said young moss seedlings are used as moss for greening.

14. The method of producing a moss plant for greening, wherein young moss seedlings obtained by the method of production set forth in claim 1 are placed on a moss mat support medium under plant factory atmosphere and reared.

15. The method of producing a moss mat, wherein moss is reared in nutrient solution in a state where said moss is retained in said mat support medium with an aperture portion at least on one surface.

16. The method of producing a moss mat, wherein moss is reared in nutrient solution irradiated with light from the normal line direction of a mat surface in a state where said moss is retained in said mat support medium with an aperture portion at least on one surface.

17. Young moss seedlings wherein said regeneration buds are bred around gametophytes of moss.

18. The young moss seedlings as set forth in claim 17, wherein enveloping surfaces of tips of said regeneration buds bred are spindle-shaped.

19. The young moss seedlings having regeneration buds with breeding directionality around gametophytes.

20. The young moss seedlings as set forth in claim 17, wherein said young moss seedlings are used as moss for greening.
